(19) United States
(12) Patent Application Publication
(10) Pub. No.: US 2023/0277358 A1
(43) Pub. Date: Sep. 7, 2023

(54) HARM BLOCKER

(71) Applicant: Sara Gomez, La Habra, CA (US)

(72) Inventor: Sara Gomez, La Habra, CA (US)

(21) Appl. No.: 17/944,049

(22) Filed: Sep. 13, 2022

Related U.S. Application Data
(60) Provisional application No. 63/243,211, filed on Sep. 13, 2021.

(51) Int. Cl. A61F 5/37

(52) U.S. Cl. CPC A61F 5/37; A61M 2021/0016

(57) ABSTRACT

An illustrated view of an exemplary harm blocker for protecting a person that has entered into a self-harming event as well as their caregiver is presented. The harm blocker is useful for preventing injury to the person during a self-harming event and to diffuse or allow the event to conclude without harm to the person or their caretaker. Furthermore, the harm blocker to be light weight, soft, reusable and easy to use. The harm blocker provides a calming effect via color and scent and distracts the afflicted person from inflicting harm upon themselves. The harm blocker further provides data for tracking impact events as well as reduces fatigue on any staff that is trying to hold or prevent the self-harming event. The harm blocker is to prevent harm for self-injury and also to prevent harm and provide protection when clients attack their caregivers.

### PRIORITY

This utility application claims the benefit of U.S. Provisional Patent Application No. 63/243,211, filed on Sep. 13, 2021, the entirety is hereby incorporated by reference.

### FIELD OF THE INVENTION

This invention relates to a person harming themselves as well as others. More particularly, it relates protective device for protecting the person from harming themselves and others.

### BACKGROUND

Self-harm or self-injury is the intentional direct injury of one's own body usually without a suicidal intention. Other terms such as head banging, cutting and self-mutilation have been used for any self-harming behavior regardless of suicidal intent. The most common form of self-harm is using a sharp object to cut the skin. Other forms include scratching, hitting, or burning body parts or hitting head or body on objects, often times hard enough to incur a concussion. While earlier usage included interfering with wound healing, excessive skin-picking, hair-pulling, and the ingestion of toxins, current usage distinguishes these behaviors from self-harm. Likewise, tissue damage from drug abuse or eating disorders is not considered self-harm because it is ordinarily an unintended side-effect.

Although self-harm is by definition non-suicidal, it may still be life-threatening. People who do self-harm are more likely to commit suicide, and self-harm is found in 40-60% of suicides. Still, only a minority of self-harmers are suicidal.

The desire to self-harm is a common symptom of some personality disorders. People with other mental disorders may also self-harm, including those with depression, anxiety disorders, substance abuse, eating disorders, post-traumatic stress disorder, schizophrenia, mental retardation, autism and dissociative disorders. Studies also provide strong support for a self-punishment function, and modest evidence for anti-dissociation, interpersonal-influence, anti-suicide, sensation-seeking, and interpersonal boundaries functions. Self-harm can also occur in high-functioning individuals who have no underlying mental health diagnosis.

The motivations for self-harm vary. Some use it as a coping mechanism to provide temporary relief of intense feelings such as anxiety, depression, stress, emotional numbness, or a sense of failure. Self-harm is often associated with a history of trauma, including emotional and sexual abuse. There are a number of different methods that can be used to treat self-harm and which concentrate on either treating the underlying causes or on treating the behavior itself. Other approaches involve avoidance techniques, which focus on keeping the individual occupied with other activities, or replacing the act of self-harm with safer methods that do not lead to permanent damage.

A shield is a piece of personal armor held in the hand, which may or may not be strapped to the wrist or forearm. Shields are used to intercept specific attacks, whether from close-ranged weaponry or projectiles such as arrows, by means of active blocks, as well as to provide passive protection by closing one or more lines of engagement during combat.

Aggression towards those that are caring for individuals with special needs is high. When a person is being physically harmful to themselves by hitting their own head, pulling out their hair, scratching their face or any other self-directed harm, staff has few if any techniques that can help prevent the self-harm. This can allow the person to inflict harm upon themselves. This can cause serious injuries, both physical and mental, as well as harm to others. Often times when the person is striking or attacking their own head, the trauma that can be caused has long term affects. When they are interrupted from engaging in these behaviors, they will often then attack the person who is attempting to protect them from themselves.

No devices are currently available that can help when a person becomes harmful to themselves other than placing the person in a strait jacket or handcuffs which is not optimal or even legal in most settings.

Accordingly, and in light of the foregoing, it would be desirable to provide harm blocker that can be used during a self-harming event to diffuse or allow the event to conclude without harm to the person or the caretaker protecting them. It would also be advantageous for the harm blocker to be light weight, soft, reusable and easy to use.

### BRIEF DESCRIPTION OF THE DRAWINGS

FIG. 1 is an illustrated back view of an exemplary harm blocker.

FIG. 2 is an illustrated view of an inside of the harm blocker shown in FIG. 1.

FIG. 3 is an illustrated back view of the harm blocker shown in FIG. 1.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

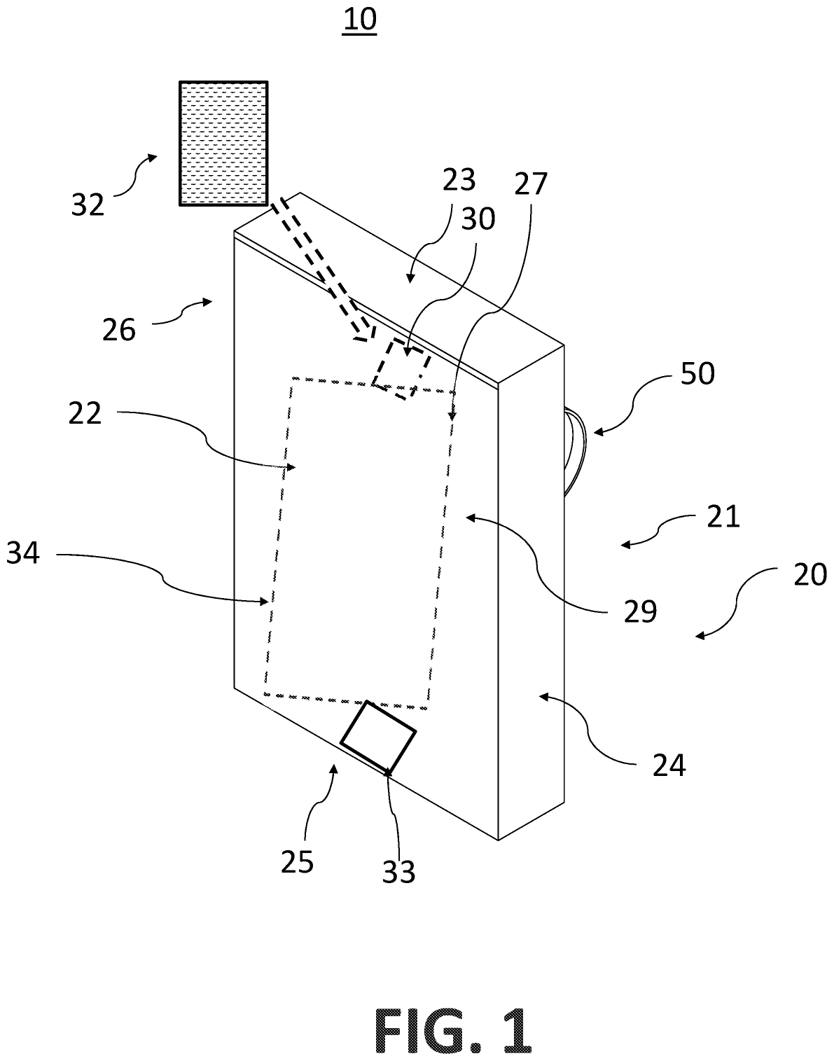

Referring to FIG. 1, an illustrated view of an exemplary harm blocker 10 for protecting a person that has entered into a self-harming event as well as their caregiver is presented. The harm blocker 10 is useful for preventing injury to the person during a self-harming event and to diffuse or allow the event to conclude without harm to the person or their caretaker. Furthermore, the harm blocker 10 to be light weight, soft, reusable and easy to use. The harm blocker provides a calming effect via color and scent and distracts the afflicted person from inflicting harm upon themselves. The harm blocker 10 further provides data for tracking impact events as well as reduces fatigue on any staff that is trying to hold or prevent the self-harming event. The harm blocker 10 is to prevent harm for self-injury and also to prevent harm and provide protection when clients attack their caregivers.

The harm blocker 10 has a body 20. The body 20 has a front 21, a back 22, a first side 23, a second side 24, a third side 25, a fourth side 26 and an inside 27. The body 20 is preferably a rectangular shape, however other shapes are hereby contemplated, including, but not limited to, square, round, oval, etc. The body 20 is preferably three (3) feet in length, however other lengths are hereby contemplated, including, but not limited to, two and one-half (2.5) feet, four (4) feet, etc. The body 20 is preferably two (2) feet in width, however other widths are hereby contemplated, including, but not limited to, one and one-half (1.5) feet, two and one-half (2.5) feet, etc. The body 20 is preferably two (2) feet in depth, however other depths are hereby contemplated, including, but not limited to, one and one-half (1.5) feet, two and one-half (2.5) feet, etc. The body 20 is preferably made of a foam material, however other materials are hereby contemplated, including, but not limited to, cotton with a bean-bag filling, denim with sand filling, etc.

The front 21 of the body 20 is preferably convex in shape, however other shapes are hereby contemplated, including, but not limited to, flat, concave, etc. The front 21 has an opening 30. The opening 30 of the front 21 of the body 20 has a hook and loop fastener, such as a Velcro®-like fastener, to open and close the opening 30 of the front 21 of the body 20. The opening 30 of the front of the body 20 may contain a scented sheet 32, such as a dryer sheet, however it may be any type of sheet of fabric or paper that can be scented. The front 21 of the body 20 is preferably perforated; however, the front 21 of the body 20 may be solid, random holes, etc.

The back 22 of the body 20 has an opening 33 and a frame 34. The opening 33 of the back 22 of the body 20 allows access to the inside 27 of the body 20. The frame 34 of the back 22 of the body 20 is extended from the front 21 of the body 20.

Each of the first side 23, the second side 24, the third side 25 and the fourth side 26 are coupled to an edging 28 of the body 20. The first side 23, second side 24, the third side 25 and the fourth side 26 are preferably solid, however the first side 23, second side 24, the third side 25 and the fourth side 26 may be flexible, corrugated, etc.

The inside 27 of the body 20 has a handle 50 that extends through a middle 29 of the body 20 to substantially near the front 21 of the body 20. The handle 50 is preferably six (6) feet in length, however other lengths are hereby contemplated, including, but not limited to, five (5) feet, eight (8) feet, etc.

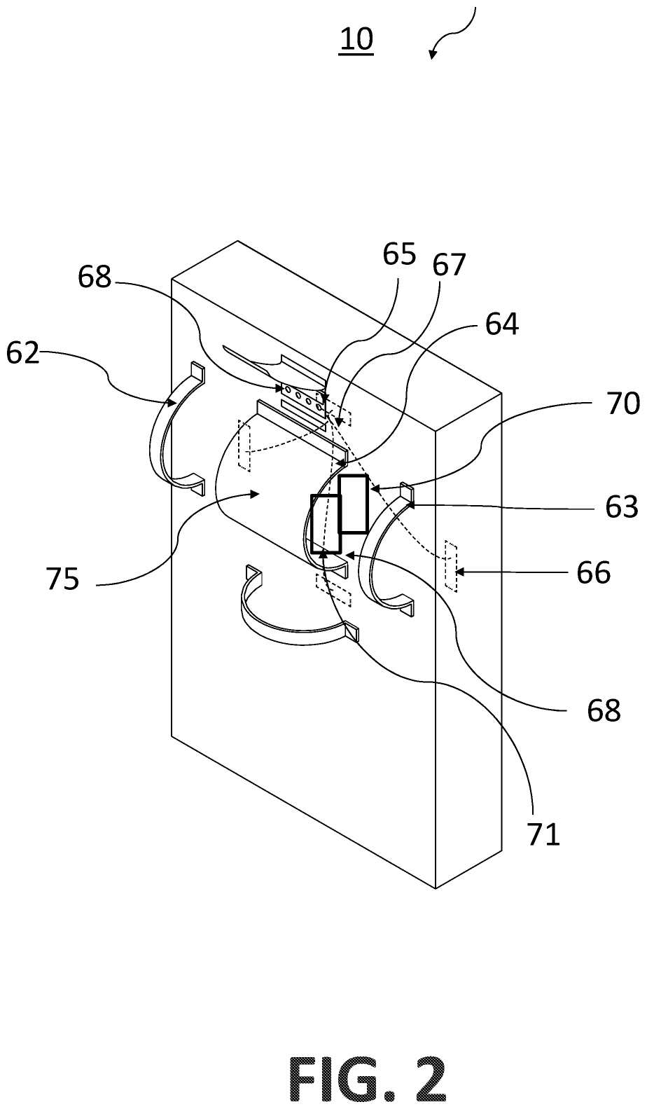

Moving now to FIG. 2, an illustrated view of the inside of the harm blocker 10 shown in FIG. 1 is presented.

The inside 27 of the harm blocker 10 has a panel 60. The panel 60 of the inside 27 of the harm blocker 10 is coupled to or part of a back side 61 of the front 21 of the body 10.

The inside 27 further has a plurality of handles 62, 63, a strap 64 and a sensor system 65. The sensor system 65 has a plurality of sensors 66, a plurality of wires 67 and a display panel 68. The display panel 69 has a computer chip 70 and a display 71. Each of the sensors 66 is coupled to the display panel 68 by one of the plurality of wires 67.

The handles 62, 63, strap 64 and sensor system 65 are coupled to the panel 60 of the inside 27 of the harm blocker 10. The sensors 66 of the sensor system 65 detect a number of contacts. The sensors 66 send the number of contacts to the computer chip 70 of the display panel 68. The computer chip 70 determines the number of contacts over a period of time and sends the number of contacts over a period of time to be displayed on the display 71 of the display panel 68.

A cover 75 is coupled to the panel 60 near a top 76 of the display panel 68 and may be extended over the display panel 68 to protect the display panel 68.

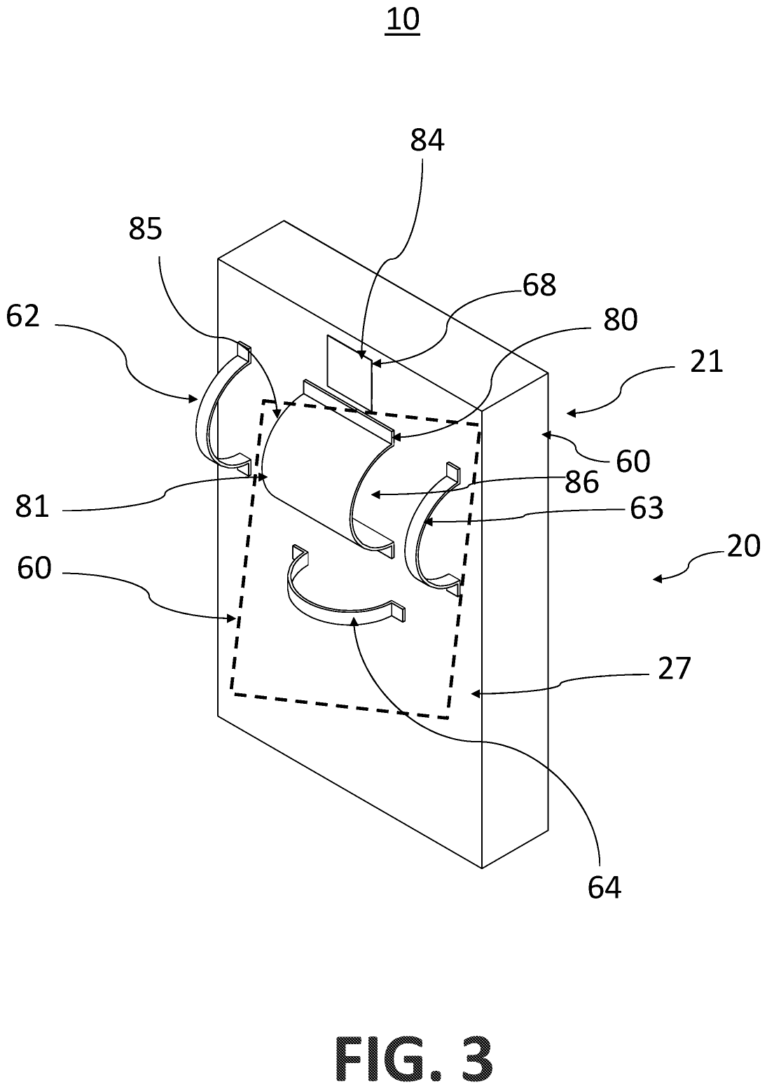

Moving now to FIG. 3, an illustrated back view of the harm blocker 10 shown in FIG. 1 is presented.

From the back 22 of the body 20, the inside 27 of the harm blocker 10 has a panel 60. The panel 60 of the inside 27 of the harm blocker 10 is coupled to or part of a back side 61 of the front 21 of the body 20.

The inside 27 further has the plurality of handles 62, 63, the strap 64, a receiving coupling 80 and the cover 81.

The handles 62, 63, the strap 64, the receiving coupling 80 and a top 83 of the cover 81 are coupled to the panel 60 of the inside 27 of the harm blocker 10.

The cover 82 is coupled to the panel 60 near a top 84 of the display panel 68 and may be extended over the display panel 68 to protect the display panel 68.

On an interior side 85 of the cover 82 is a first coupler 86. The first coupler 86 is configured to be coupled to the receiving coupler 80 so as to secure a bottom 87 of the cover 82 to the panel 60. The first coupler 86 is preferably a hook and loop fastener, such as a Velcro-like fastener, however other types of fasteners are hereby contemplated, including, but not limited to snap, zipper, two-sided tape, etc. The receiving coupler 80 is preferably a hook and loop fastener, such as a Velcro-like fastener, however other types of fasteners are hereby contemplated, including, but not limited to snap, zipper, two-sided tape, etc.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A harm blocker for protecting a person that has entered into a self-harming event, the harm blocker comprising:
a body, the body comprising:
a first side, a second side, a third side and a fourth side, wherein the first side, the second side, the third side and the fourth side form a frame.
a front, the front being coupled to a first end of the frame;
a back, the back having an inside, the inside being hollow and the inside being configured substantially in a middle of the back, the inside having a panel, wherein the panel being configured to be coupled to an inside of the front;
a plurality of handles, the handles being coupled to the back of the panel of the inside;
a sensor system, the sensor system being coupled to the panel of the inside, the sensor system comprising:
a display panel, the display panel having a computer chip and a display;
a plurality of wires;
a plurality of sensors, each of the plurality of sensors being coupled to the computer chip by one of the plurality of wires.
a cover, a top of the cover being coupled to the panel of the inside, an inside of the cover having a first coupling substantially near the bottom of the cover, wherein the cover being for protecting the display panel; and
a receiving coupling, the receiving coupling being configured to be coupled to the first coupling of the cover.

2. The harm blocker of claim 1, the body being made of a foam material.

3. The harm blocker of claim 1, wherein the body have a rectangular shape.

4. The harm blocker of claim 1, wherein the first coupler is a hook and loop fastener.

5. The harm blocker of claim 1, wherein the receiving coupler is a hook and loop fastener.

6. The harm blocker of claim 1, wherein the body has a length of three feet.

7. The harm blocker of claim 1, wherein the body has a width of two feet.

8. The harm blocker of claim 1, wherein the body has a depth of two feet.

9. The harm blocker of claim 1, wherein the front of the body has a convex shape.

10. The harm blocker of claim 1, wherein the front of the body has a scented sheet.

11. The harm blocker of claim 10, wherein the scented sheet is a dryer sheet.

12. The harm blocker of claim 1, wherein the front of the body has a round shape.

13. The harm blocker of claim 1, wherein the fourth is solid.

14. The harm blocker of claim 1, wherein the third side is solid.

15. The harm blocker of claim 1, wherein the first side is flexible.

16. The harm blocker of claim 1, wherein the second side is flexible.

17. The harm blocker of claim 1, wherein the inside of the body has a handle.

18. The harm blocker of claim 17, wherein the handle extends through a middle of the body to the front of the body.

19. The harm blocker of claim 1, wherein the front of the body is perforated.

20. The harm blocker of claim 1, wherein the front of the body is solid.
